(19) United States
(12) Patent Application Publication
(10) Pub. No.: US 2026/0096879 A1
(43) Pub. Date: Apr. 9, 2026

(54) REPLACEMENT HEART VALVE WITH PLEATED OUTER SKIRT

(71) Applicant: BOSTON SCIENTIFIC SCIMED, INC., MAPLE GROVE, MN (US)

(72) Inventor: Gregory Dyer, Maple Grove, MN (US)

(73) Assignee: BOSTON SCIENTIFIC SCIMED, INC., MAPLE GROVE, MN (US)

(21) Appl. No.: 19/353,049

(22) Filed: Oct. 8, 2025

Related U.S. Application Data
(60) Provisional application No. 63/705,171, filed on Oct. 9, 2024.

(51) Int. Cl. A61F 2/24

(52) U.S. Cl. CPC A61F 2/2418; A61F 2/2415; A61F 2220/0075; A61F 2240/001; A61F 2250/0039

(57) ABSTRACT

A replacement heart valve frame assembly includes an expandable framework and an outer skirt coupled to an outer surface of the expandable framework. The outer skirt includes a plurality of pleats.

### CROSS-REFERENCE TO RELATED APPLICATIONS

This application claims the benefit of priority of U.S. Provisional Application No. 63/705,171 filed Oct. 9, 2024, the entire disclosure of which is hereby incorporated by reference.

### TECHNICAL FIELD

The present disclosure pertains to medical devices, systems, and methods for manufacturing and/or using medical devices and/or systems. More particularly, the present disclosure pertains to a replacement heart valve frame assembly.

### BACKGROUND

A wide variety of intracorporeal medical devices have been developed for medical use, for example, intravascular use. Some of these devices include guidewires, catheters, replacement heart valves, medical device systems, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example replacement heart valve frame assembly includes an expandable framework, and an outer skirt coupled to an outer surface of the expandable framework, the outer skirt having a plurality of pleats.

Alternatively or additionally to the embodiment above, the plurality of pleats is configured to expand when the expandable framework is deployed.

Alternatively or additionally to any of the embodiments above, the outer skirt has a closed bottom fixed to a lower edge of the expandable framework defining an inflow end, and a free edge forming an opening facing an outflow end of the expandable framework.

Alternatively or additionally to any of the embodiments above, the outer skirt is sewn to the expandable framework and the plurality of pleats are sewn closed with at least one line of horizontal stitches extending circumferentially around the expandable framework, wherein each of the plurality of pleats is a vertical pleat extending from the line of horizontal stitches to the free cdgc.

Alternatively or additionally to any of the embodiments above, the plurality of pleats of the outer skirt are formed when the outer skirt is sewn to the expandable framework.

Alternatively or additionally to any of the embodiments above, a diameter of the free edge of the outer skirt is larger than a diameter of the expandable framework adjacent the free edge.

Alternatively or additionally to any of the embodiments above, the expandable framework includes a plurality of struts defining a lattice structure, the plurality of struts defining a plurality of lower crowns proximate an inflow end at a lower edge of the lattice structure, a plurality of upper crowns proximate an outflow end of the lattice structure, and a plurality of stabilization arches extending downstream from the outflow end of the lattice structure.

Alternatively or additionally to any of the embodiments above, a lower edge of the outer skirt is adjacent the inflow end of the expandable framework, and the outer skirt extends at least to the plurality of upper crowns.

Alternatively or additionally to any of the embodiments above, the replacement heart valve frame assembly further includes an inner skirt coupled to an inner surface of the expandable framework, wherein the inner skirt is coupled to the outer skirt adjacent the inflow end.

Alternatively or additionally to any of the embodiments above, the replacement heart valve frame assembly further includes a plurality of valve leaflets coupled to the expandable framework.

Another example replacement heart valve frame assembly includes an expandable framework comprising a plurality of struts defining a lattice structure having a central lumen, the plurality of struts defining a plurality of lower crowns defining an inflow end of the lattice structure, a plurality of upper crowns proximate an outflow end of the lattice structure, and a plurality of stabilization arches extending downstream from the outflow end of the lattice structure, and an outer skirt disposed on an outer surface of the lattice structure, the outer skirt having a closed upstream end fixed to the lattice structure, the outer skirt having a free edge forming a single circumferential opening between the expandable framework and an inner surface of the outer skirt, the free edge facing the outflow end of the lattice structure, wherein a diameter of the free edge of the outer skirt is larger than a diameter of the expandable framework.

Alternatively or additionally to the embodiment above, the free edge of the outer skirt extends downstream at least to the plurality of upper crowns.

An example method of manufacturing a replacement heart valve frame assembly includes providing a frame, and coupling an outer skirt to the frame, wherein the outer skirt includes a plurality of vertical pleats.

Alternatively or additionally to the embodiment above, coupling the outer skirt to the frame involves a single step of forming the plurality of vertical pleats simultaneously with sewing the outer skirt to the frame.

Alternatively or additionally to the embodiment above, coupling the outer skirt to the frame includes first sewing the plurality of vertical pleats in the outer skirt, and then after the plurality of vertical pleats are sewn, coupling the outer skirt to the frame.

Alternatively or additionally to the embodiment above, the method further includes forming the outer skirt with a tapered configuration, wherein the outer skirt has a larger diameter at its upper edge compared to its lower edge.

Alternatively or additionally to the embodiment above, forming the outer skirt comprises using a blow molding process to create the plurality of vertical pleats in the outer skirt.

Alternatively or additionally to the embodiment above, the blow molding process includes placing a piece of skirt material in a female mold with recesses, inserting a male mold over the piece of skirt material, the male mold being smaller than the female mold, and inflating the piece of skirt material to create excess material that forms the plurality of vertical pleats.

Alternatively or additionally to the embodiment above, the female mold has an outer circumferential wall surrounding an inner lumen, wherein the outer circumferential wall includes a plurality of undulations.

Alternatively or additionally to the embodiment above, the male mold is a cylinder.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIGS. 1A and 1B illustrate prior art replacement heart valve implants;

FIG. 2. illustrates an example replacement heart valve implant with a pleated outer skirt;

FIGS. 3A-3C illustrate steps in an example method of forming a pleated outer skirt;

FIGS. 4A and 4B illustrate steps in an example method of attaching a pleated outer skirt to an expandable framework; and

FIGS. 5A-5C illustrate another example method of forming a pleated outer skirt on an expandable framework.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate example embodiments of the disclosure but not limit the disclosure. Those skilled in the art will recognize that the various elements described and/or shown may be arranged in various combinations and configurations without departing from the scope of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently-such as, but not limited to, angularly, radially, circumferentially (e.g., along an arc), etc. Additionally, the term “substantially” when used in reference to two dimensions being “substantially the same” shall generally refer to a difference of less than or equal to 5%.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Diseases and/or medical conditions that impact the cardiovascular system are prevalent throughout the world. Some mammalian hearts (e.g., human, etc.) include four heart valves: a tricuspid valve, a pulmonary valve, an aortic valve, and a mitral valve. Some relatively common medical conditions may include or be the result of inefficiency, ineffectiveness, or complete failure of one or more of the valves within the heart. For example, failure of the aortic valve or the mitral valve can have a serious effect on a human and could lead to a serious health condition and/or death if not dealt with properly. Treatment of defective heart valves poses challenges in that the treatment often requires the repair or outright replacement of the defective heart valve. Disclosed herein is an apparatus, system, and/or method that may be used in a portion of the cardiovascular system in order to diagnose, treat, and/or repair the system. In some embodiments, the apparatus, system, and/or method disclosed herein may be used before and/or during a procedure to diagnose, treat, and/or repair a defective heart valve (e.g., the aortic valve, the mitral valve, etc.). In addition, a replacement heart valve implant may be delivered percutaneously and thus may be much less invasive to the patient. The apparatus, system, and/or method disclosed herein may also provide other desirable features and/or benefits as described below.

It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to “the leaflet”, “the strut”, or other features may be equally referred to all instances and quantities beyond one of said feature unless clearly stated to the contrary. As such, it will be understood that the following discussion may apply equally to any and/or all of the components for which there are more than one within the replacement heart valve implant and/or the apparatus unless explicitly stated to the contrary.

Additionally, it should be noted that in any given figure, some features may not be shown, or may be shown schematically, for clarity and/or simplicity. Additional details regarding some components and/or method steps may be illustrated in other figures in greater detail. The systems, devices, and/or methods disclosed herein may provide a number of desirable features and benefits as described in more detail below. For the purpose of this disclosure, the discussion below is directed toward the treatment of a native aortic valve and will be so described in the interest of brevity. This, however, is not intended to be limiting as the skilled person will recognize that the following discussion may also apply to a mitral valve or another heart valve with no or minimal changes to the structure and/or scope of the disclosure. Similarly, the medical devices disclosed herein may have applications and uses in other portions of a patient's anatomy, such as but not limited to, arteries, veins, and/or other body lumens.

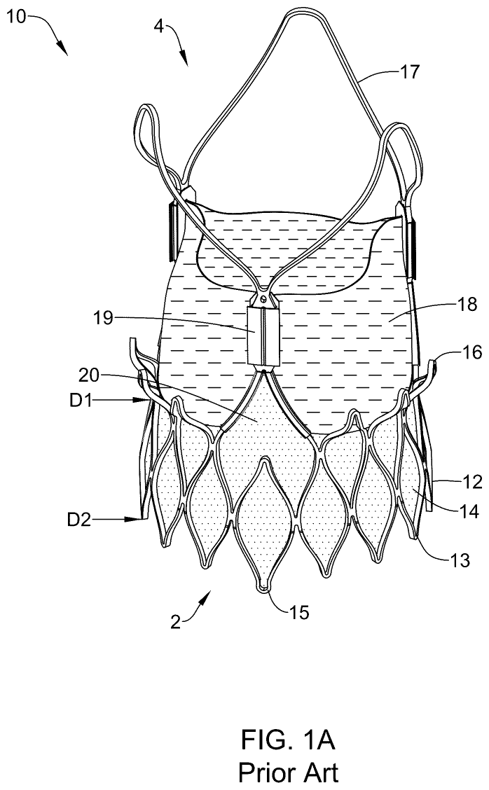

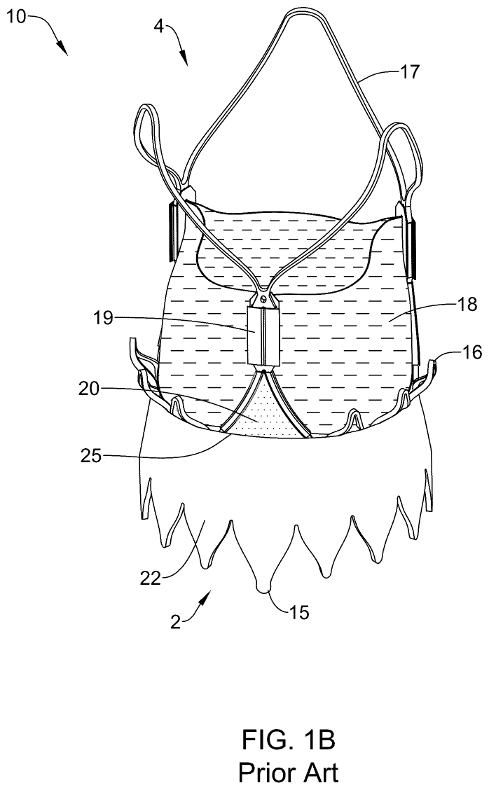

Prosthetic or replacement heart valves are medical devices used to replace damaged or diseased natural heart valves. These valves typically consist of an expandable frame and a valve assembly. FIG. 1A illustrates a prior art replacement heart valve implant 10 allowing one-way flow therethrough from an inflow end 2 to an outflow end 4. The replacement heart valve implant 10 includes an expandable framework 12 defining a central lumen. The expandable framework 12 includes a plurality of struts 13 defining a lattice structure disposed and/or extending around a central longitudinal axis. The plurality of struts 13 defines a plurality of interstices 14 (e.g., openings) between adjacent frame struts and/or through the expandable framework 12. The expandable framework 12 defines a plurality of lower crowns 15 at the inflow end 2 of the lattice structure, a plurality of upper crowns 16 proximate an outflow end of the lattice structure, and a plurality of stabilization arches 17 extending downstream from the outflow end of the lattice structure. The plurality of stabilization arches 17 extends downstream of and/or away from the upper crowns 16 in a direction opposite the lower crowns 15.

The replacement heart valve implant 10 includes a plurality of valve leaflets 18 disposed within a central lumen of the expandable framework 12. The plurality of valve leaflets 18 are coupled, secured, and/or fixedly attached to the expandable framework 12. The expandable framework 12 includes a plurality of commissures 19 disposed at a base of the plurality of stabilization arches 17, joining circumferentially adjacent stabilization arches. The replacement heart valve implant 10 and/or the expandable framework 12 may include an inner skirt 20 attached to the inner surface of the expandable framework 12, and an outer skirt 22 attached to the outer surface of the expandable framework 12, as shown in FIG. 1B. The outer skirt 22 is not shown in FIG. 1A in order to illustrate the underlying structure of the expandable framework 12.

The outer skirt 22 may play an important role in sealing the replacement heart valve implant against the patient's anatomy to prevent paravalvular leakage. Previous approaches to reducing paravalvular leakage have included various skirt structures, such as standard cylindrical skirts, as shown in FIG. 1, and those with multiple ribs. However, these structures often face challenges in conforming to irregular anatomies and maintaining a low profile during delivery. When the outer skirt does not conform to the patient's anatomy, leakage may occur between the replacement heart valve and the patient's′ anatomy. The present invention overcomes these limitations with a pleated skirt structure that provides additional material to conform to the patient's anatomy, regardless of how irregular it is.

The expandable framework 12 may be provided in at least four sizes: small(S), medium (M), large (L), and extra-large (XL). These frame sizes may also be referred to using a numerical designation of 23, 25, 27, and 29, respectively. The number refers to an average outer diameter of the expandable framework 12. The portion of the expandable framework 12 between the lower crowns 15 and the upper crowns 16 is generally slightly tapered, with the outer diameter D1 just below the upper crowns 16 being smaller than the outer diameter D2 at the lower crowns 15. See FIG. 1A. The standard and currently used outer skirt is generally formed on a mandrel having the same diameter as D2. Due to the outer skirt being formed from a flat piece of material that is molded into a ring structure, the ring may be slightly tapered with one edge having a slightly larger diameter than the opposite edge. The conventional way of affixing the outer skirt 22 to the expandable framework 12 is to fasten the wider edge of an outer skirt 22 to the lower, wider, portion of an expandable framework 12 that is the same size as the outer skirt 22, such as adjacent the lower crowns 15 at D2, and fasten the narrower edge of the outer skirt just below the upper crowns 16, at the narrower part of the expandable framework 12 at D1. In this manner, the silhouette of the outer skirt 22 and the expandable framework 12 generally match.

In one embodiment, the dimensions of the outer skirt 22 relative to the expandable framework 12 may be altered to achieve an increased volume of outer skirt material that may provide enhanced sealing and conformity against the patient's anatomy. A larger diameter free downstream edge 25 of the outer skirt 22 may be achieved by coupling an outer skirt 22 sized larger than the expandable framework 12. For example, a size extra-large outer skirt may be coupled to a size medium or large expandable framework, a size large outer skirt may be coupled to a size small or medium expandable framework, or any other combination in which the outer skirt diameter is larger than the outer diameter of the expandable framework. Examples of expandable framework outer diameter are small at 23 mm, medium at 25 mm, large at 27 mm, and extra-large at 29 mm.

In another embodiment, the increased volume of outer skirt material may be achieved by inverting the outer skirt 22 relative to the expandable framework 12 prior to attachment, and/or a larger mandrel may be used to form the outer skirt 22. In this manner, the widest part of the outer skirt 22 would be adjacent the upper crowns 16, where the outer diameter D1 of the expandable framework 12 is smallest, while the narrowest part of the outer skirt 22 would be adjacent the lower crowns 15 where the outer diameter D2 of the expandable framework 12 is larger. As shown in Table 1 below, the standard outer skirt (OS) mandrel is generally 1.0 mm larger than the frame size, providing a close fit of the outer skirt 22 over the expandable framework 12. Using a larger OS mandrel that is at least 2.0 mm larger than the frame size may achieve an outer skirt 22 with an outer diameter at least 2.0 mm larger than the expandable framework at the D1 location just under the upper crowns 16. This increase in outer skirt material at the D1 location may aid in sealing the replacement heart valve implant against the patient's anatomy.

TABLE 1

| | | | | | Standard | Maximum Outer Skirt | | |
|---|---|---|---|---|---|---|---|---|
| | | | | | Outer | Dimensions | | |
| | | D1 | | D2 | Skirt | Larger | | |
| | | dimensions | | dimensions | (OS) | Max OS | OS Max | OS Min |
| | Frame | Min | Max | Greater | OS | Mandrel | Diam | Diam |
| Size | Size | (mm) | (mm) | than (mm) | Mandrel | Size | (Typical) | (Typical) |
| Small (S) | 23 | 22.7 | 23.7 | 24 | 24 | 25 | 25.9 | 25 |
| Medium (M) | 25 | 24.7 | 25.7 | 26 | 26 | 27 | 27.9 | 27 |
| Large (L) | 27 | 26.7 | 27.7 | 28 | 28 | 29 | 29.9 | 29 |
| Extra-large | 29 | 28.8 | 29.8 | 30 | 30 | 31 | 31.9 | 31 |
| (XL) | | | | | | | | |

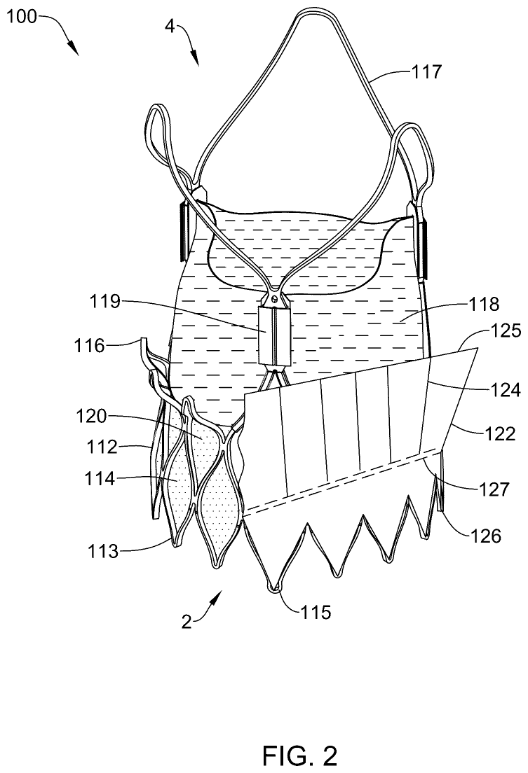

In another embodiment, a replacement heart valve implant 100 may have a pleated outer skirt 122, as shown in FIG. 2. The pleated outer skirt 122 is illustrated partially cut-away in order to show the underlying structure of the expandable framework 112. It should be appreciated that the replacement heart valve implant 100 can be any type of heart valve (e.g., a mitral valve, an aortic valve, etc.). The replacement heart valve implant 100 allows one-way flow through the replacement heart valve implant 100 from the inflow end 2 to the outflow end 4. After implantation, the outer skirt 122 will be disposed between the expandable framework 112 and the vessel wall in order to prevent fluid, such as blood, from flowing around the replacement heart valve implant 100 and/or the expandable framework 112 in a downstream direction. The outer skirt 122 may ensure the fluid flows through the replacement heart valve implant 100 only in the downstream direction from inflow end 2 to outflow end 4, and does not flow around the replacement heart valve implant 100, causing regurgitation. This ensures that the plurality of valve leaflets 118 attached within the expandable framework 112 can stop the flow of fluid when in the closed position.

The replacement heart valve implant 100 includes an expandable framework 112 defining a central lumen. The expandable framework 112 is configured to shift from a collapsed configuration to an expanded configuration. The expandable framework 112 may be self-expanding and self-biased toward the expanded configuration. Alternatively, the expandable framework 112 may be mechanically expandable. The expandable framework 112 includes a plurality of struts 113 defining a lattice structure disposed and/or extending around a central longitudinal axis. The plurality of struts 113 defines a plurality of interstices 114 (e.g., openings) between adjacent struts 113 and/or through the expandable framework 112. The expandable framework 112 defines a plurality of lower crowns 115 at the inflow end 2 of the lattice structure, a plurality of upper crowns 116 proximate an outflow end of the lattice structure, and a plurality of stabilization arches 117 extending downstream from the outflow end of the lattice structure. The plurality of stabilization arches 117 extends downstream of and/or away from the upper crowns 116 in a direction opposite the lower crowns 115.

A plurality of valve leaflets 118 are coupled, secured, and/or fixedly attached to the expandable framework 112 and disposed within the central lumen of the expandable framework. Each of the plurality of valve leaflets 118 may include a root edge coupled to the expandable framework 112 and a free edge (e.g., a coaptation edge) movable relative to the root edge to coapt with the coaptation edges of the other leaflets along a coaptation region. The expandable framework 112 includes a plurality of commissures 119 disposed at a base of the plurality of stabilization arches 117, joining circumferentially adjacent stabilization arches. Between circumferentially adjacent commissures 119, the replacement heart valve implant 100 may be devoid of the expandable framework 112 at a longitudinal position radially outward of the free edges of the plurality of valve leaflets 118. As such, the free edges of the plurality of valve leaflets 118 may be free from direct contact with the expandable framework 112 as the plurality of valve leaflets 118 open and/or close.

The replacement heart valve implant 100 may include an inner skirt 120 fixed to and extending along an inner surface of the expandable framework 112 between the expandable framework and the valve leaflets 118, and the outer skirt 122 coupled to and extending along an outer surface of the expandable framework 112. The inner skirt may be fixed to the outer skirt 122 adjacent the inflow end of the expandable framework. In some embodiments, the inner skirt 120 may be coupled to the lower crowns 115 and/or the upper crowns 116. In some embodiments, the inner skirt 120 may be coupled only to the upper crowns 116.

In some embodiments, the inner skirt 120 and/or the outer skirt 122 may seal one of, some of, a plurality of, or all of the plurality of interstices 114 formed in the expandable framework. In at least some embodiments, sealing the interstices may be considered to prevent fluid from flowing through the interstices from inside of the expandable framework 112 to outside of the expandable framework 112. In some embodiments, the inner skirt 120 and/or the outer skirt 122 may be attached to the expandable framework 112 and/or the plurality of struts 113 using one or more methods including but not limited to tying with sutures or filaments, adhesive bonding, melt bonding, embedding or over molding, welding, etc.

The outer skirt 122 may include a plurality of folds or pleats 124. The pleats 124 may be vertical and configured to expand when the replacement heart valve implant 100 is deployed. This expansion of the pleats 124 allows the outer skirt 122 to fill gaps between the expandable framework 112 and the patient's anatomy, effectively reducing or preventing paravalvular leakage. During delivery, the pleated outer skirt structure allows for a reduced profile of the replacement heart valve implant 100, facilitating easier insertion and navigation through the patient's vasculature. The pleats 124 may be engineered to expand into areas around the replacement heart valve implant 100 where the expandable framework 112 may not fully expand due to anatomical variations or calcification. This adaptive expansion of the outer skirt 122 may enhance the replacement heart valve implant's 100 sealing capabilities. The entirety of the outer skirt 122, including all pleats 124, may be formed as a single, monolithic structure.

The upper, downstream edge of the outer skirt 122 may be a free edge 125 forming an opening facing the outflow end of the expandable framework, as shown in FIG. 2. The free edge 125 may be devoid of any attachment to the expandable framework 112. The outer skirt 122 may be sewn to the expandable framework 112 and the plurality of pleats 124 may be sewn closed at the upstream end with at least one row of horizontal stitches 127 extending circumferentially around the expandable framework. In some embodiments, two lines of stitches 127 may be positioned offset from one another such that the stitches in one line are positioned circumferentially offset from the stitches in the other line of stitches 127, as shown in FIG. 2. The outer skirt 122 may be devoid of any attachment to the expandable frame downstream of the line(s) of stitches 127. The resulting structure of the outer skirt 122 may define only a single circumferential opening, pocket or channel that extends around the entire circumference of the expandable framework 112, and extends radially between the outer surface of the expandable framework 112 and the inner surface of the outer skirt 122, and also extends axially between the line of stitches 127 and the free edge 125. The line(s) of stitches 127 may be continuous to form a closed bottom of the plurality of pleats 124 and prevent any fluid that enters the outer skirt 122 in an upstream direction from flowing through the outer skirt 112 or around the outer skirt 122 to a position upstream of the outer skirt 122. Each of the plurality of pleats 124 may be a vertical pleat extending from the closed bottom at the line(s) of stitches 127 to the free edge 125. The line(s) of stitches 127 may form the vertical pleats 122 as box pleats, knife pleats, inverted box pleats, or a combination thereof.

The outer skirt 122 may be sewn to the expandable framework 112 at both the inflow end struts 113 and with at least one line of circumferential stitches 127. In other embodiments, the outer skirt 122 may be attached to the expandable framework 112 only with the at least one line of circumferential stitches 127, and the lower, upstream edge of the outer skirt 122 may be cut immediately upstream of the at least one line of circumferential stitches 127, without covering the lower crowns 115.

The outer skirt 122 may have a tapered shape, with a larger diameter at its free upper edge 125 compared to its lower edge 126. The diameter of the outer skirt free upper edge 125 may be larger than the diameter of the expandable framework 112 adjacent the free upper edge 125. This tapered structure results in additional outer skirt material positioned around the outside of the expandable framework 112, enhancing the outer skirt's 122 ability to conform to the patient's anatomy and prevent leakage. In general, the outer diameter of the free upper edge 125 of the pleated outer skirt 122 in the folded configuration may be at least 2.0 mm larger than the outer diameter of the expandable framework 112. In some embodiments, the diameter of the pleated outer skirt at the free upper edge 125, with the pleats 124 in their folded configuration, may be between 25 mm and 32 mm, as compared to an outer diameter of the expandable framework 112 of between 23 mm and 29 mm, depending on whether the expandable framework 112 is size small, medium, large, or extra-large. When the pleats 124 of the outer skirt 122 unfold, the outer diameter at the free upper edge 125 will be significantly larger than in the folded configuration, for example between 1.25 to 3 times the outer diameter when the pleats 124 are in their folded configuration.

The outer skirt 122 may be coupled to the lower crowns 115. In some embodiments, the outer skirt 122 may have a lower edge 126 fixed to the lower edge of the expandable framework defining the inflow end 2. This creates a closed lower edge 126 of the outer skirt 122. The outer skirt 122 may be coupled to the lower crowns 115 with sutures following the zig-zag pattern of struts 113 defining the lower crowns 115 at the inflow end of the expandable framework 112. The outer skirt 122 may extend downstream at least to the upper crowns 116. In some embodiments, the outer skirt 122 may cover the upper crowns 116 and extend toward the commissures 119, as illustrated in FIG. 2.

A method of manufacturing a replacement heart valve frame assembly may include providing an expandable framework 112 and coupling an outer skirt 122 to the frame, where the outer skirt includes a plurality of pleats 124. The replacement heart valve frame assembly may then have a valve assembly coupled to an inner surface of the frame to form the replacement heart valve implant 100. The frame may be an expandable framework 112 as described above, and the valve assembly may include the valve leaflets 118 described above. The step of coupling the outer skirt 122 to the expandable framework 112 may include sewing the outer skirt 122 to the expandable framework 112 and forming the plurality of pleats 124.

The outer skirt 122 may be coupled to the expandable framework 112 using either of two primary methods for creating the plurality of pleats 124 in the outer skirt 122. The plurality of pleats 124 of the outer skirt 122 may be formed when the outer skirt is sewn to the expandable framework. This single step sewing method may involve forming the plurality of pleats 124 simultaneously with sewing the outer skirt 122 to the expandable framework 112. In this method, the plurality of pleats 124 may be formed at the same time the outer skirt 122 is sewn to the expandable framework 112 with at least one row of circumferential stitches 127. If the outer skirt 122 extends over the lower crowns 115, the outer skirt 122 may then be sewn to the struts 113 forming the lower crowns 115, after the pleats 124 have been formed.

In another embodiment, the plurality of pleats 124 may be formed in the outer skirt 122 before the outer skirt is sewn to the expandable framework 112. This two-step method involves first sewing the plurality of pleats 124 in the outer skirt 122, and then after the pleats are sewn, coupling the pleated outer skirt 122 with the expandable framework 112. After the plurality of pleats 124 are sewn in the outer skirt 122, the outer skirt 122 may be sewn to the expandable framework 112, with either or both of at least one row of circumferential stitches 127 and sewing the lower edge of the outer skirt 122 to the struts 113 forming the lower crowns 115.

In either the single step or two step methods, an inner skirt 120 may be coupled to the expandable framework and/or the outer skirt 122. In any method of making the replacement heart valve frame assembly, the outer skirt 122 may be formed with a tapered configuration, where the outer skirt has a larger diameter at its free upper edge 125 compared to its lower edge 126. The outer skirt 122 may be formed with a frustonconical shape having a central lumen.

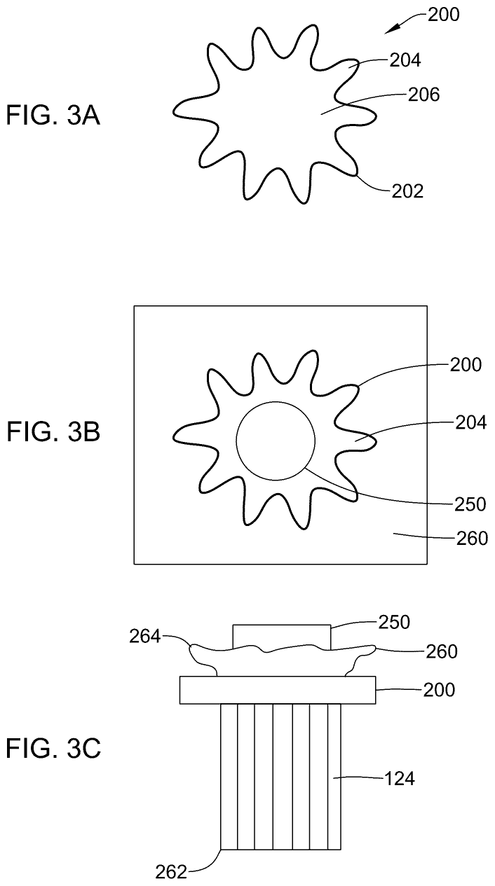

One method of manufacturing the pleats 124 in the outer skirt 122 involves a blow molding process, illustrated in FIGS. 3A, 3B, and 3C. This process may use a female mold 200 with a plurality of recesses 204 and a smaller male mold 250. The female mold 200 may have an outer circumferential wall 202 surrounding an inner lumen 206, where the outer circumferential wall 202 includes a plurality of undulations forming the plurality of recesses 204, as shown in FIG. 3A. The male mold 250 may be a cylinder.

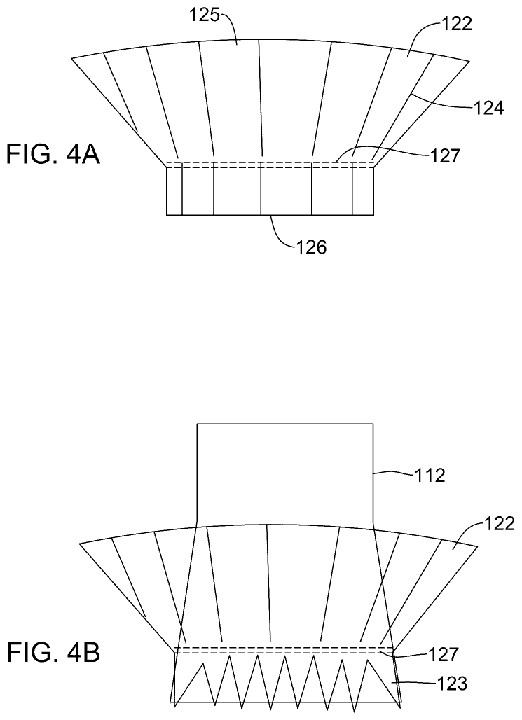

The method may include placing a flat piece of skirt material 260 over the female mold 200 and then inserting the male mold 250 over the skirt material 260, as shown in FIG. 3B. As the male mold 250 is pushed into the female mold 200, the skirt material 260 may be inflated, creating excess material that forms the pleats 124, as shown in FIG. 3C. The blow molding process naturally creates pleats when the male mold 250 is loose on the female mold 200 and when the piece of skirt material 260 is loose during blow molding. This process of converting a flat piece of skirt material 260 into a generally cylindrical shape often results in the formed skirt having a frustonconical shape instead of a perfect cylinder. This may be due to the piece of skirt material 260 stretching and narrowing at the closed end 262 contacting the male mold 250 as compared to the free end 264. The resulting outer skirt 122 from the above-described blow molding process may have the plurality of pleats 124 sewn down by two rows of circumferential stitches 127 adjacent one end. In some examples the rows of stitches 127 may be positioned adjacent the free end 264. The closed end 262 may then be cut off, allowing the pleats 124 to open up, as shown in FIG. 4A.

Alternatively, the pleats 124 in the outer skirt 122 may be formed by manually folding the skirt material and sewing down the pleats 124 with at least one row of circumferential stitches 127. The pleats 124 may continue to the lower edge 126 of the outer skirt 122, as shown in FIG. 4A. In other embodiments, the outer skirt 122 may terminate immediately below the row(s) of stiches 127. In the two-step method of forming the replacement heart valve frame assembly that may then have valve leaflets attached to form the replacement heart valve implant 100, a plurality of pleats 124 are sewn into the outer skirt 122 before the outer skirt 122 is coupled to the expandable framework 112. The pleated outer skirt 122 may then be coupled to the expandable framework 112, as shown in FIG. 4B. Additional rows of circumferential stitches 127 may be used to sew the pleated outer skirt 122 to the expandable framework 112. the row(s) of circumferential stitches 127 may extend across the interstices above the lower crowns 115. The lower portion 123 of the outer skirt 122 may also be coupled to the lower struts of the expandable framework. Alternatively, the lower portion 123 of the outer skirt 122 may be removed.

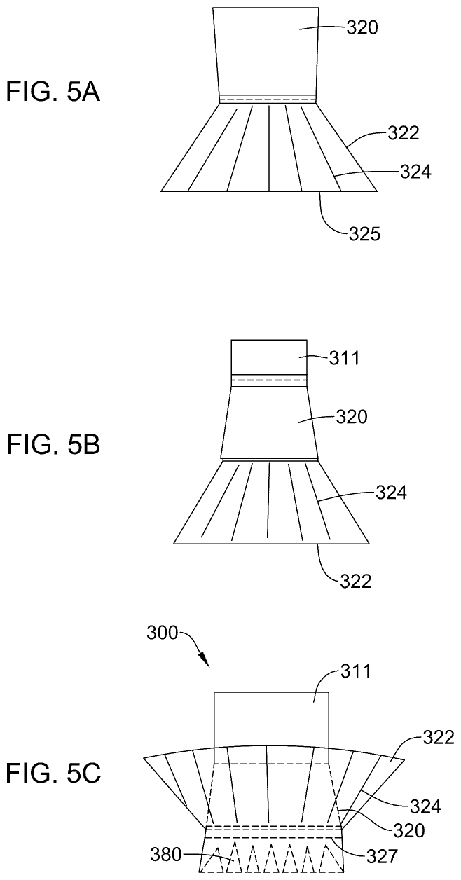

FIGS. 5A, 5B, and 5C illustrate another method of forming a replacement heart valve frame assembly that may have a plurality of valve leaflets attached thereto to form a replacement heart valve implant 300 with a pleated outer skirt 322. In this method, a non-pleated inner skirt 320 is attached to an outer skirt 322 that has a plurality of pleats 324 sewn into it, forming a conjoined skirt. The outer skirt 322 may be pleated using the blow molding process described above, or the pleats may be manually sewn into the outer skirt 322. The outer skirt 322 is sewn to the inner skirt 320 such that the larger diameter of the outer skirt is the free edge 325, as shown in FIG. 5A, with the narrower edges of the inner skirt 320 and the outer skirt 322 sewn together. The inner skirt 320 is then inserted into the inner lumen of an expandable framework 312 from the inflow end adjacent the lower crowns, and sewn to the inner surface of the expandable framework 312, as shown in FIG. 5B. The pleated outer skirt 322 is then folded up onto the lower portion of the expandable framework 312, and at least one row of circumferential stitches 327 is sewn through the outer skirt 322 to the expandable framework 312 and inner skirt 320, as shown in FIG. 5C. Excess material 380 of the inner and outer skirts 320, 322 may be cut out between lower crowns. It will be understood that the method illustrated in FIGS. 5A-5C may be used with a non-pleated outer skirt. A non-pleated outer skirt would be attached to the inner skirt such that the larger diameter of the tapered non-pleated outer skirt was the free edge.

The inverted and conjoined outer skirt structures described above have been evaluated for regurgitation flow (RF) performance compared to standard designs in which an outer skirt is fixed to a frame of the same size with the outer skirt in the same orientation as the frame. The primary metric used to quantify performance improvements was Regurgitation Flow (RF), expressed as a percentage. Results showed that the inverted and conjoined skirt structures consistently demonstrated lower RF values compared to standard designs across different sizes. In one study, an example conventional XL outer skirt on an XL frame exhibited an RF % of 92.8 while an XL outer skirt inverted on an XL frame exhibited an RF % of 48.2.

In another study, conventional small outer skirts on small frames exhibited an RF % of 87.9, 87.6, and 87.1, while a conjoined inverted medium outer skirt on a small frame exhibited an RF % of 46.9 and a conjoined inverted small outer skirt on a small frame exhibited an RF % of 48. Overall, the inverted and conjoined outer skirts showed a reduction of total RF % of approximately 50%.

The outer skirt 122, 322 may be made from various materials, including porcine or bovine tissue, with the pleated structure potentially allowing for similar performance to PET (polyethylene terephthalate) skirts. The pleated construction of the outer skirt is compatible with existing delivery systems, allowing for the outer skirt to be crimped along with the rest of the replacement heart valve implant 100 into a small enough package to be tracked and delivered to the implant site.

The materials that can be used for the various components of the medical device system and the various elements thereof disclosed herein may include those commonly associated with medical devices. For simplicity purposes, the following discussion refers to the system. However, this is not intended to limit the devices and methods described herein, as the discussion may be applied to other elements, members, components, or devices disclosed herein, such as, but not limited to, the expandable framework, the inner skirt, the outer skirt, the plurality of leaflets, and/or elements or components thereof.

In some embodiments, the plurality of valve leaflets 118 may be comprised of a polymer, such as a thermoplastic polymer. In some embodiments, the plurality of valve leaflets 118 may include at least 50 percent by weight of a polymer. In some embodiments, the plurality of valve leaflets 118 may be formed from bovine or porcine pericardial or other living tissue. Other configurations and/or materials are also contemplated. The inner skirt 120 may include a polymer, such as a thermoplastic polymer. In some embodiments, the inner skirt 120 may include at least 50 percent by weight of a polymer. In some embodiments, the outer skirt may include a polymer, such as a thermoplastic polymer. In some embodiments, the outer skirt may include at least 50 percent by weight of a polymer. In some embodiments one or more of the plurality of valve leaflets 118, the inner skirt 120, and/or the outer skirt 122, 322 may be formed of the same polymer or polymers. In some embodiments, the polymer may be a polyurethane. In some embodiments, the inner skirt 120 and/or the outer skirt 122, 322 may be substantially impervious to fluid. In some embodiments, the inner skirt 120 and/or the outer skirt 122, 322 may be formed from a thin tissue (e.g., bovine or porcine pericardial tissue, etc.). In some embodiments, the inner skirt 120 and/or the outer skirt 122, 322 may be formed from a coated fabric material. In some embodiments, the inner skirt 120 and/or the outer skirt 122, 322 may be formed from a nonporous and/or impermeable fabric material. Other configurations are also contemplated. Some suitable but non-limiting examples of materials that may be used to form the inner skirt and/or the outer skirt including but not limited to polymers, composites, and the like, are described below.

In some embodiments, the additional system and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylenc/poly(alkylene ether) phthalate and/or other polyester elastomers such as HYTREL® available from DuPont), polyamide (for example, DURETHAN® available from Bayer or CRISTAMID® available from Elf Atochem), clastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfonc, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS 50A), polycarbonates, polyisobutylene (PIB), polyisobutylene polyurethanc (PIBU), polyurethanc silicone copolymers (for example, Elast-Eon® from AorTech Biomaterials or ChronoSil® from AdvanSource Biomaterials), ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-clastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof, may be made of a material that does not substantially distort the image and create substantial artifacts (i.e., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A replacement heart valve frame assembly, comprising:
an expandable framework; and
an outer skirt coupled to an outer surface of the expandable framework, the outer skirt having a plurality of pleats.

2. The replacement heart valve frame assembly of claim 1, wherein the plurality of pleats is configured to expand when the expandable framework is deployed.

3. The replacement heart valve frame assembly of claim 2, wherein the outer skirt has a closed bottom fixed to a lower edge of the expandable framework defining an inflow end, and a free edge forming an opening facing an outflow end of the expandable framework.

4. The replacement heart valve frame assembly of claim 3, wherein the outer skirt is sewn to the expandable framework and the plurality of pleats are sewn closed with at least one line of horizontal stitches extending circumferentially around the expandable framework, wherein each of the plurality of pleats is a vertical pleat extending from the line of horizontal stitches to the free edge.

5. The replacement heart valve frame assembly of claim 4, wherein the plurality of pleats of the outer skirt are formed when the outer skirt is sewn to the expandable framework.

6. The replacement heart valve frame assembly of claim 3, wherein a diameter of the free edge of the outer skirt is larger than a diameter of the expandable framework adjacent the free edge.

7. The replacement heart valve frame assembly of claim 1, wherein the expandable framework includes a plurality of struts defining a lattice structure, the plurality of struts defining a plurality of lower crowns proximate an inflow end at a lower edge of the lattice structure, a plurality of upper crowns proximate an outflow end of the lattice structure, and a plurality of stabilization arches extending downstream from the outflow end of the lattice structure.

8. The replacement heart valve frame assembly of claim 7, wherein a lower edge of the outer skirt is adjacent the inflow end of the expandable framework, and the outer skirt extends at least to the plurality of upper crowns.

9. The replacement heart valve frame assembly of claim 8, further comprising an inner skirt coupled to an inner surface of the expandable framework, wherein the inner skirt is coupled to the outer skirt adjacent the inflow end.

10. The replacement heart valve frame assembly of claim 1, further comprising a plurality of valve leaflets coupled to the expandable framework.

11. A replacement heart valve frame assembly comprising:
an expandable framework comprising a plurality of struts defining a lattice structure having a central lumen, the plurality of struts defining a plurality of lower crowns defining an inflow end of the lattice structure, a plurality of upper crowns proximate an outflow end of the lattice structure, and a plurality of stabilization arches extending downstream from the outflow end of the lattice structure; and
an outer skirt disposed on an outer surface of the lattice structure, the outer skirt having a closed upstream end fixed to the lattice structure, the outer skirt having a free edge forming a single circumferential opening between the expandable framework and an inner surface of the outer skirt, the free edge facing the outflow end of the lattice structure, wherein a diameter of the free edge of the outer skirt is larger than a diameter of the expandable framework.

12. The replacement heart valve frame assembly of claim 11, wherein the free edge of the outer skirt extends downstream at least to the plurality of upper crowns.

13. A method of manufacturing a replacement heart valve frame assembly, comprising:
providing a frame; and
coupling an outer skirt to the frame, wherein the outer skirt includes a plurality of vertical pleats.

14. The method of claim 13, wherein coupling the outer skirt to the frame involves a single step of forming the plurality of vertical pleats simultaneously with sewing the outer skirt to the frame.

15. The method of claim 13, wherein coupling the outer skirt to the frame includes first sewing the plurality of vertical pleats in the outer skirt, and then after the plurality of vertical pleats are sewn, coupling the outer skirt to the frame.

16. The method of claim 13, further comprising forming the outer skirt with a tapered configuration, wherein the outer skirt has a larger diameter at its upper edge compared to its lower edge.

17. The method of claim 16, wherein forming the outer skirt comprises using a blow molding process to create the plurality of vertical pleats in the outer skirt.

18. The method of claim 17, wherein the blow molding process comprises:
placing a piece of skirt material in a female mold with recesses;
inserting a male mold over the piece of skirt material, the male mold being smaller than the female mold; and
inflating the piece of skirt material to create excess material that forms the plurality of vertical pleats.

19. The method of claim 18, wherein the female mold has an outer circumferential wall surrounding an inner lumen, wherein the outer circumferential wall includes a plurality of undulations.

20. The method of claim 34, wherein the male mold is a cylinder.
